(19) United States
(12) Patent Application Publication
(10) Pub. No.: US 2006/0211748 A1
(43) Pub. Date: Sep. 21, 2006

(54) Sulphydryl compounds in combination with sulpha compounds

(76) Inventors: Allen I. Bain, Vancouver (CA); Alexander Zolotoy, Richmond (CA); Jesse D. Raffa, Vancouver (CA)

Correspondence Address: NIELDS & LEMACK, 176 EAST MAIN STREET, SUITE 7, WESTBORO, MA, 01581, US

(21) Appl. No.: 10/520,395

(22) PCT Filed: Jul. 11, 2003
(86) PCT No.: PCT/IB03/03538

§ 371 Date: Jan. 23, 2006

Related U.S. Application Data
(60) Provisional application No. 60/395,170, filed on Jul. 11, 2002.

(51) Int. Cl. A61K 31/426; A61K 31/385; A61K 31/198; A61K 31/135

(52) U.S. Cl. 514/369; 514/440; 514/562; 514/646

(57) ABSTRACT

The present invention discloses various compositions and methods for co-administration of one or more sulfa compounds or pharmaceutically acceptable salts thereof, and one or more sulphydryl compounds in the treatment or prevention of disease, wherein the disease has in whole or part microbial and/or inflammatory origins.

This application claims priority of Ser. No. 60/395,170 filed on Jul. 11, 2002, the disclosure of which is hereby incorporated by reference.

### FIELD OF THE INVENTION

The present invention is related to the treatment or prevention of microbial or inflammatory diseases by co-administration of one or more sulpha compounds with one or more sulphydryl compounds.

### BACKGROUND TO THE INVENTION

Certain specific sulphones, sulphonamides, and derivatives are useful as human and veterinary therapeutics as anti-microbial agents, anti-inflammatory agents, or both. In patients in need of this therapy, the routine administration of these drugs is, however, often not permitted due to the serious adverse effects of the drugs. In some cases, practitioners do not prescribe these drugs due to the risk of such side effects. In other cases, patients may begin therapy successfully, but are forced to terminate therapy prematurely due to the seriousness of side effects. For example, despite the efficacy of DDS in the treatment of leprosy, its side effects have led to such serious toxicity, that drug developers have brought to market a wide variety of new agents; some of these are less effective, but less toxic. In the treatment of opportunistic infections associated with AIDS, the toxicity of DDS results in discontinuation of clinical use by about 25% of patients. In patients suffering from dementia, the use of DDS has been suggested by retrospective data (U.S. Pat. No. 5,532,219). However, it is well known to those skilled in the art that the use of DDS in patients suffering from dementia, such as for example those with HIV infection and the elderly, is fraught with the dangers of toxicity and such use has therefore never been approved by regulatory agencies nor by most clinicians.

Much of the toxicity that is caused by sulphones such as DDS, and by sulphonamides such as sulphamethoxazole, has been demonstrated to be due to the toxic potential of certain metabolites. For example, for DDS and sulphamethoxazole, it is known that cytochrome enzymes in the liver can catalyze the conversion of the parent compound to its respective N-hydroxylamine and that this metabolite is oxidized by the oxygen in the presence of haemoglobin, to a N-nitroso derivative, hydrogen peroxide and methaemoglobin (Kiese 1974). Both the hydroxylamine and the nitroso metabolites have been demonstrated to have toxic potential. Hydroxylamines of primary amines cause hemolytic anemia (Jollow 1995, Grossman et al 1988, Reilly et al 1999) and methaemoglobinemia (Coleman et al 1996). Hydroxylamines of secondary amines do not form nitroso compound under oxidation and, accordingly, are associated primarily with hemolytic anemia (primaquine, hydroxychloroquine, diclofenac and others).

While some attention has been directed to reduction of the toxicity of DDS and related sulphone and sulphonamide agents, many such agents have simply fallen into disfavor as a result of their inherent toxicity and the availability of superior, less toxic drugs. For example, the use of cimetidine to inhibit the catalytic action of liver cytochromes, thereby partially preventing metabolism of certain drugs into their toxic N-hydroxylamine derivatives, has been suggested (Coleman, 1995). However, such use of cimetidine is not expected to be acceptable in old patients, in whom: a) cimetidine may exacerbate pre-existing dementia (Basavaraju, 1980), b) cimetidine is not recommended to use more than 400 mg/day whereas significant reduction of DDS metabolism demands about 1200 mg/day.

N-acetyl-L-cysteine has been studied extensively as an agent that can absorb a variety of free radicals and can cause an increase in glutathione content. However, since N-acetyl-L-cysteine has a relatively short half-life after oral administration and cannot be utilized to produce additional glutathione, this agent has not been considered by most clinicians to have specific therapeutic activity. Indeed, even though low glutathione levels measured in certain patients infected with HIV have been normalized, at least partially, by administration of N-acetyl-L-cysteine; the use of this agent in HIV-infected patients is not consistently associated with improvement in clinically relevant endpoints (James, 1996; Anonymous, 1995).

Since N-acetyl-L-cysteine has been shown to increase glutathione, it would be expected to help avoid toxicity only where a lack of glutathione permits the toxicity to develop. Reilly et al (2000), however, suggest that DDS toxicity may not be as dependent on glutathione levels as is the toxicity of sulphonamides.

Additionally, the metabolism of DDS within the erythrocyte has, been shown to be cyclic, with the hydroxylamine metabolite of DDS binding oxyhaemoglobin and forming the nitroso derivative, followed by glutathione-dependent regeneration of hydroxylamine. According to this reaction sequence, the state of the art would indicate that treatments that result in enhanced glutathione levels would result in enhanced cycling of the metabolite and consequent development or exacerbation of methaemoglobinemia.

Finally, a synergistic effect of a combination of DDS, DDS metabolites and N-acetyl-L-cysteine on inflammatory targets is not obvious. DDS inhibits killing of neurons in assay Aβ1-42-microglia and inhibit neutrophil adherence (Moldschielder et al 2000, Thuong-Nguen V et al 1993). It is suggested that inhibition of neutrophil adherence is associated with inhibition of chemoattractant-induced signal transduction (Debol S. M. 1997). DDS metabolites, DDS hydroxylamine and DDS N-chloramine are formed by activated neutrophils, monocytes, lymphocytes (Uetrecht 1992, 1988). Both metabolites are capable to the direct chemical reactions and inactivation of inflammatory targets. Thus Naisbitt et al (1999) demonstrated that the hydroxylamine and nitroso metabolites of sulphamethoxazole are responsible for inhibition of neutrophil function, and that the parent sulphonamide had no effect. Thus if N-acetyl-L-cysteine counteracts adverse effects of metabolites by the neutralization of metabolites, then anti-inflammatory activity of the combination is not obvious. N-acetyl-L-cysteine is a precursor of a stable radical and one must mention that a recent study of vitamin E (precursor of free radical) against Alzheimer disease was successful for a special subgroup (JAMA, 2002, Jun. 26). Such a combination of potential agents evidently is not obvious invention to one skilled in the art.

Lastly, the state of art did not indicate any example both in vivo and in vitro where NAC was related with the most important characteristics of hemolytic anemia, i.e. half-life of red cells or haemoglobin formation

Surprisingly, we found that N-acetyl-L-cysteine limits certain adverse side effects of DDS, when orally administered with DDS, while not reducing the efficacy or potency of DDS but instead acting synergistically. N-acetyl-L-cysteine is one representative of a class of compounds that contains the sulphydryl radical and can be co-administered with certain sulphones and sulphonamides to prevent some important manifestations of the toxicity of the latter and to act synergistically with it, respectively referred to herein as “Sulphydryl Compound” and “Sulpha Drug”.

### SUMMARY OF THE INVENTION

4,4′-diaminodiphenylsulphone (DDS) and related drugs are widely used for either their anti-microbial effects or their anti-inflammatory effects, or both, and other beneficial effects. However this class of drugs is well known for a number of adverse effects with a main contribution of hemolytic anemia and methaemoglobinemia.

While many agents have been shown to prevent the formation or toxic activity of the toxic metabolites of DDS and related drugs in vitro only a few, such as cimetidine, have been suggested to be somewhat useful in the clinical situation. The present invention provides a system and method for administering DDS and related drugs with agents that will permit the beneficial activity while blocking the formation or action of the toxic metabolites of these drugs.

### DETAILED DESCRIPTION OF THE INVENTION

The present invention provides a system and a method for utilizing certain pharmacodynamic and pharmacokinetic characteristics of a Sulphydryl Compound such that said compound is effective in preventing toxicity of a Sulpha Drug, in particular hemolytic anemia. In order to be effective in the invention, the Sulphydryl Compound may be administered by one of various routes nearly simultaneously with the administration of the Sulpha Drug by the same route. Possible routes are not limited by oral, intravenous, topical and parenteral administrations with a preferable oral administration. The two respective agents are synergistic in their actions when administered as described herein, as well as in dosage regimens that are alternated.

The compound that can serve as a Sulphydryl Compound includes any compounds known to contribute intracellular cysteine toward the synthesis of glutathione, any compounds that contain the sulphydryl radical, their precursors, isomers, analogs and derivatives, pharmaceutically acceptable salts, stereoisomers, metabolites, metabolic precursors or prodrugs of these systems in either crystalline, or amorphous, or liquid or gel forms. A preferable sulphydryl compound may be any one of or combination of N-acetyl-L-cysteine, 2-oxathiozolidine carboxylate, cysteine, cysteamine, alpha-lipoic acid and dihydrolypoic acid.

The compound that can serve as a Sulpha Drug in the present invention includes compounds with the sulphone or sulphonamide and amine moieties, adjacent to the aromatic fragment, including isomers, stereoisomers, analogs, pharmaceutically acceptable salts, metabolites, metabolic precursors or prodrugs of these systems in either crystalline or amorphous or liquid or gel forms.

A system for ensuring that the Sulphydryl Compound is orally administered at the appropriate temporal proximity to the oral administration of the Sulpha Drug includes any formulation, packaging system, printed protocol, or combination thereof, that facilitates or encourages the appropriate dosing behavior by the patient, the caregiver, or both.

The pharmaceutical compositions of the present invention are useful in effectively treating numerous conditions, including Alzheimer disease, dementia, AIDS dementia, AIDS pneumonia, asthma, malaria, dermatitis herpetiformis, Chronic Obstructive Pulmonary Disease, Amyotrophic Lateral Sclerosis, rheumatoid arthritis, linear IgA bullous dermatosis, treat subcorneal pustular dermatoses, benign chronic bullous disease of childhood, bullous eruptions of systemic lupus erythematosus, pemphigus, pemphigoid, erythema elevatum diutinum, Sweet's syndrome, granuloma faciale, Henoch-Schonlein purpura, pyoderma gangrenosum, hypocomplementic urticarial vasculitus, rheumatoid vasculitus, discoid lupus, systemic lupus erythematosus, cutaneous manifestation of systemic lupus erythematosus, panniculitus, relapsing polychondritis, acne, alopecia mucinosa, pustular psoriasis, brown recluse spider bites, Parkinson's disease, multiple sclerosis, adverse effects caused by head trauma, adverse effects of hemorrhage caused by head trauma, encephalitis, meningitis, Kaposi sarcoma, Bechet's disease, and Creutzfeldt Jakob Disease.

The dose-range of Sulpha Drug is between 20 to 350 mg/day. A preferable dose of Sulpha Drug is 100 mg/day. A preferable number of administrations of the Sulfa Drug is 2 times per day, 50 mg each. The dose-range of Sulphydryl Compound is between 300 to 6000 mg/day. A preferable dose of sulphydryl compound is between 1200 to 2000 mg/day. The preferable number of administrations of sulphydryl compounds is 4 times per day from 300 to 500 mg each.

Combination of Sulpha Drug and Sulphydryl Compound (“Combination”) of the presented invention should be administered in association with one or more inert carriers, excipients and/or diluents. Assayable amounts of Combination of the invention will generally vary from about 0.001% to about 75% wt % of the entire weight of the composition. Preferred oral composition contains between 0.1% and about 50% of the Combination. Preferred parenteral dosage includes between 0.01 to 10% by weight of the Combination. A preferred topical formulation contains a concentration of the Combination of from 0.1 to about 25% w/v (weight per unit volume).

Inert carriers include any material that does not degrade or otherwise covalently react with a compound of the invention.

Solid composition for oral administration may include: binders such as syrups, acacia, sorbitol, polyvinylpyrrolidone, carboxymethylcellulose, ethyl cellulose, microcrystalline cellulose or gelatin and mixtures thereof; excipients like starch, lactose or dextrins, disintegrating agents as alginic acid, sodium alginate, Primogel and the like; lubricants as magnesium stearate, heavy molecular weight acids as stearic acid, high molecular weight polymers such as polyethylene glycol; sweetening agents as sucrose or saccharine; a flavoring agent such as peppermint, methyl salicylate or orange flavoring, and a coloring agent.

The liquid pharmaceutical compositions of the invention, whether they be solutions, suspensions or other like form may include: sterile diluents such as water for injection, saline solution preferably physiological saline, Ringer's solution, isotonic sodium chloride, fixed oils such as synthetic mono or diglycerides which may serve as the solvent or suspending medium, polyethylene glycols, glycerin, propylene glycol or other solvents. The compounds may be in the form of the free base or in the form of a pharmaceutically acceptable salt such as hydrochloride, sulfate, citrate, fumarate, methanesulfonate, acetate, tartrate, maleate, lactate, mandelate, salicylate and other salts known in the art.

### EXAMPLE 1

A capsule containing 600 mg of N-acetyl-L-cysteine together with excipients and a tablet containing 100 mg of DDS are placed side-by-side in each of seven wells formed in a plastic film. Said plastic film and its wells is then covered with a second film which is adhesively affixed to the first film so as to hold the seven pairs of capsules and tablets in their respective wells. The plastic film is marked such that each well is designated with a different day of the week in sequence.

### EXAMPLE 2

A capsule is filled with 600 mg of N-acetyl-L-cysteine and a tablet containing 100 mg of DDS is placed inside the same capsule. The remaining space in said capsule is filled with excipient grade sugar prior to assembly of the two halves of the capsule.

### EXAMPLE 3

DDS in its pharmaceutical grade powder form, and L-2-oxothiazolidine-4-carboxylate in its pharmaceutical grade powder form are thoroughly admixed in a mass ratio of five to one. The resulting mixture is further admixed with colloidal silicone dioxide, magnesium stearate, microcrystalline cellulose, and cornstarch. This latter mixture is then pressed into tablets with a tableting machine.

### EXAMPLE 4

A drug container is constructed with two compartments that are side-by-side but not interconnected. Each compartment is large enough to contain 20 capsules of N-acetyl-L-cysteine or 20 tablets of DDS. The open end of the compartments of the drug container are covered with a three position closure, one position which allows for dispensing of DDS tablets, one position which allows for dispensing of N-acetyl-L-cysteine capsules, and one position which maintains the drug container in a closed configuration.

### EXAMPLE 5

A drug container containing DDS tablets and another drug container containing N-acetyl-L-cysteine capsules are packaged together in a plastic wrapper that is clearly labeled with the words “always take one drug from each container and administer by mouth as closely together as practicable”.

### EXAMPLE 6

Blood is drawn from laboratory rats into heparinized tubes, washed using conventional methods, and resuspended in buffered saline solution. Red blood cells are labeled with ⁵¹Cr as chromate using the CPD standard method known to those skilled in the art. After resuspension in isotonic saline, DDS N-hydroxylamine alone (control) or with N-acetyl-L-cysteine (treatment) are added at final concentrations of 0.05 to 0.5 millimolar and 2 millimolar to 10 millimolar, respectively, and incubated for two hours. A 0.5 milliliter sample of the washed and resuspended red blood cells is then reintroduced into the autologous animal. Half-life of the labeled erythrocytes is determined by blood sampling every two days and counting of radioactive decay in the sample using a gamma counter. N-acetyl-L-cysteine is able to preserve the normal half-life of ⁵¹Cr-labelled erythrocytes challenged in vitro with DDS N-hydroxylamine and re-introduced to autologous rats.

### EXAMPLE 7

Thirty individuals are randomized to receive administered 100 mg DDS per day either alone with 1000 mg N-acetyl-L-cysteine for 8 weeks. Prior to the trial, patients are stratified by genetic polymorphism for glucose-6-dehydrogenase enzyme activity, age, haemoglobin concentration, and a general health score. Blood is drawn prior to and every week after commencement of dosing and haemoglobin concentration, half-life of red cells, hematocrit, reticulocyte count and bilirubin is analyzed. Parameters of hemolysis are compared between matched cohorts of treatment (DDS with N-acetyl-L-cysteine) and control (DDS alone) patients, and for the entire treatment group and the entire control group. The treatment group has significantly less evidence of hemolysis in the majority of treatment group cohorts in compared with matched control group cohorts.

We claim:

1. A composition comprising one or more sulphone or sulphonamide compounds and/or their respective isomers, analogs, derivatives, stereoisomers, metabolites, metabolic precursors, prodrugs in the crystalline, amorphous, liquid or gel forms and pharmaceutically acceptable salts thereof, and one or more compounds comprising the sulphydryl radical and/or their respective isomers, analogs, derivatives, stereoisomers, metabolites, metabolic precursors, prodrugs in the crystalline, amorphous, liquid or gel forms and pharmaceutically acceptable salts thereof.

2. The composition of claim 1, wherein said sulphone or sulphonamide compounds are selected from the group consisting of sulphone or sulphonamide and amine moieties adjacent to the aromatic group, and wherein said compounds comprising said sulphydryl radical are selected from the group consisting of N-acetyl-L-cysteine, L-2-oxothiazolidine-4-carboxylate, cysteine, cysteamine, alpha-lipoic acid and diortholipoic acid.

3. A composition according to claim 1 wherein said sulphone compound is 4,4′-diaminodiphenylsulphone.

4. A composition according to claim 1 wherein said compound containing the sulphydryl radical is N-acetyl-L-cysteine.

5. A method of administering a composition according to claim 1, wherein the mode of administration is selected from the group consisting of parenteral injection, nasal administration, transdermal administration, rectal administration, inhaled administration, topical administration and oral administration.

6. A preparation for oral administration in which 4,4′-diaminodiphenylsulphone and N-acetyl-L-cysteine are admixed.

7. A preparation according to claim 6 wherein the admixture is carried out in a mass ratio of between 2 and 20 times the mass of N-acetyl-L-cysteine as that of 4,4′-diaminodiphenylsulphone.

8. A kit comprised of a dosage form of an effective amount of one or more sulphone or sulphonamide compounds and/or their respective isomers, analogs, derivatives, stereoisomers, metabolites, metabolic precursors, prodrugs in the crystalline, amorphous, liquid or gel forms and pharmaceutically acceptable salts thereof, and one or more compounds comprising the sulphydryl radical and/or their respective isomers, analogs, derivatives, stereoisomers, metabolites, metabolic precursors, prodrugs in the crystalline, amorphous, liquid or gel forms and pharmaceutically acceptable salts thereof.

9. A kit according to claim 8 wherein one or more pairs of an oral dosage form unit of each of drugs are presented in proximity to one another and affixed to a backing film by a second film, labeled as required to facilitate patient compliance with the prescribed dosing regimen.

10. A kit according to claim 9 wherein the drugs are 4,4′-diaminodiphenylsuphone and N-acetyl-L-cysteine.

11. A kit according to claim 8 wherein said kit provides the systematic increase of the dose of one or more of the combined drugs in said kit to improve the safety or efficacy of the drug components.

12. A kit according to claim 8 wherein said kit provides the systematic decrease of the dose of one or more of the combined drugs in said kits to improve the safety or efficacy of the drug components.

13. A kit according to claim 11 wherein the systematic increase occurs over a period of 5 days to about 100 days.

14. A method of treating a disease wherein inflammation and/or microbes contribute to the pathology, comprising administering to a patient in need thereof a composition comprising one or more sulphone or sulphonamide compounds and/or their respective isomers, analogs, derivatives, stereoisomers, metabolites, metabolic precursors, prodrugs in the crystalline, amorphous, liquid or gel forms and pharmaceutically acceptable salts thereof, and one or more compounds comprising the sulphydryl radical and/or their respective isomers, analogs, derivatives, stereoisomers, metabolites, metabolic precursors, prodrugs in the crystalline, amorphous, liquid or gel forms and pharmaceutically acceptable salts thereof.

15. The method of claim 14 wherein said disease is selected from the group consisting of Alzheimer disease, dementia, AIDS dementia, AIDS pneumonia, asthma, malaria, dermatitis herpetiformis, Chronic Obstructive Pulmonary Disease, Amyotrophic Lateral Sclerosis, rheumatoid arthritis, linear IgA bullous dermatosis, treat subcorneal pustular dermatoses, benign chronic bullous disease of childhood, bullous eruptions of systemic lupus erythematosus, pemphigus, pemphigoid, erythema elevatum diutinum, Sweet's syndrome, granuloma faciale, Henoch-Schonlein purpura, pyoderma gangrenosum, hypocomplementic urticarial vasculitus, rheumatoid vasculitus, discoid lupus, systemic lupus erythematosus, cutaneous manifestation of systemic lupus erythematosus, panniculitus, relapsing polychondritis, acne, alopecia mucinosa, pustular psoriasis, brown recluse spider bites, Parkinson's disease, multiple sclerosis, adverse effects caused by head trauma, adverse effects of hemorrhage caused by head trauma, encephalitis, meningitis, Kaposi sarcoma, Bechet's disease, and Creutzfeldt Jakob Disease.

16. A method of treating a disease wherein inflammation and/or microbes contribute to the pathology, comprising co-administering to a patient in need thereof a composition comprising one or more sulphone or sulphonamide compounds and/or their respective isomers, analogs, derivatives, stereoisomers, metabolites, metabolic precursors, prodrugs in the crystalline, amorphous, liquid or gel forms and pharmaceutically acceptable salts thereof, and a composition comprising one or more compounds comprising the sulphydryl radical and/or their respective isomers, analogs, derivatives, stereoisomers, metabolites, metabolic precursors, prodrugs in the crystalline, amorphous, liquid or gel forms and pharmaceutically acceptable salts thereof.

17. The method of claim 16, wherein said disease is selected from the group consisting of Alzheimer disease, dementia, AIDS dementia, AIDS pneumonia, asthma, malaria, dermatitis herpetiformis, Chronic Obstructive Pulmonary Disease, Amyotrophic Lateral Sclerosis, rheumatoid arthritis, linear IgA bullous dermatosis, treat subcorneal pustular dermatoses, benign chronic bullous disease of childhood, bullous eruptions of systemic lupus erythematosus, pemphigus, pemphigoid, erythema elevatum diutinum, Sweet's syndrome, granuloma faciale, Henoch-Schonlein purpura, pyoderma gangrenosum, hypocomplementic urticarial vasculitus, rheumatoid vasculitus, discoid lupus, systemic lupus erythematosis, cutaneous manifestation of systemic lupus erythematosus, panniculitus, relapsing polychondritis, acne, alopecia mucinosa, pustular psoriasis brown recluse spider bites, Parkinson's disease multiple sclerosis, adverse effects caused by head trauma, adverse effects of hemorrhage caused by head trauma, encephalitis, meningitis, Kaposi sarcoma, Bechet's disease, and Creutzfeldt Jakob Disease.
